SEQUENCE LISTING (3 sequences)

SEQ ID NO: 1 - DNA, 25 bp, Artificial Sequence
  synthetic primer
gtcaccaatatggaccaggcaacgc

SEQ ID NO: 2 - DNA, 25 bp, Artificial Sequence
  synthetic primer
ccgcgtctagatgctaattgagggg

SEQ ID NO: 3 - DNA, 1331 bp, Ralstonia sp.U2
  gene (267)...(1172): nagR gene
gtcaccaatatggaccaggcaacgcaacagaacgcggcgctggtcgagcagatggccgccgcggcctccagactgaagggtcagtccgaagagctggtgcagacggtggcggtgttcaaactggcggacgatggcagttccgacccggctgcctacgcacaagaccagtcgcatgggagaactgcgccgcaccggttggcagaccaccgacatcctgatcctcagccatggccgaccgcggcagtcaatatgggtttgctggaagcttatgcttcagagaaaagctcgacgaacaactgacgtagccacatgttgcccggatcccggttgtacttggcatgccaaaacaggttgatggcgatgtcgggcagcttggccgggtgcggggatgtcgtcagaccaaaaggcacttcgcagcgaacggcaaaacgctgcggcacggtcgcgatgaggtcggtgctgtgcagaatggggccgatcgcaatgaaatgcggcaccaccagccgcatgcgccttttgatgcctgcgcgttcgagcaggccatcgacctcaccgtgtccggtgttgagtgcgaccacgccgacatgctccagttcactgaactgtttcaggctcatgggggatttggcgcttggatggtccttgcggaacatgcatacgtagcggtggcgaaagaggcgccgctggaagaatccggtctgtagctctggcagaagacccaaggcgagatcaaccgcaccggactccatatcctccttcagattgccagcattcgggcgcagcgtgctgatctggatgtgaggagctcgttgcgcaagcgcttccatcagtgggggcatgaagtacatctcgccgatgtcggtcattgccaagttgaaggtgcgcgtgctggcaaatgggtcgaaagagtcacgggtcgtcagtgccgtctgcagcgtgttgagcgcatagatcacgggctccgcaagatgcagtgcatacggtgtcggctccatgccttttgaggtgcgcaagaacaaatcgtcctttagcgccgcacgcagccgtttaagtgaattgctgacggcaggctgcgtcagccccagtttttcgccggccgtcgatacgctccggtcgagcagtagctggttgaagaccaccagcagattcaagtcgatgtcgcgcagatccatgatgcctcaccattattcatgctggtgattttaactatcagacttgatctatagcgctataccgatcgacgcgccagaatcgcagccattcggagacaactgaaaaaagagcttgcatggaactggtagtagaacccctcaattagcatctagacgcgg

(19) United States
(12) Patent Application Publication
(10) Pub. No.: US 2004/0241708 A1
(43) Pub. Date: Dec. 2, 2004

(54) Recombinant vector for transforming strain to detect benzoic acid and derivatives thereof, transformant containing the recombinant vector, and method for detecting benzoic acid and derivatives thereof using the transformant

(76) Inventors: Man-Bock Gu, Kwangju-shi (KR); Robert J. Mitchell, Kwangju-shi (KR)

Correspondence Address: KNOBBE MARTENS OLSON & BEAR LLP, 2040 MAIN STREET, FOURTEENTH FLOOR, IRVINE, CA, 92614, US

(21) Appl. No.: 10/694,546

(22) Filed: Oct. 27, 2003

(30) Foreign Application Priority Data
May 30, 2003 (KR) 2003-0034915

(51) Int. Cl. C12Q 1/68; C12N 15/74; C12N 1/21

(52) U.S. Cl. 435/6; 435/252.33; 435/488

(57) ABSTRACT

Disclosed herein is a recombinant vector for transforming a strain to detect benzoic acid and derivatives, comprising a bioluminescent gene encoding a bioluminescent protein and a gene set inducing the expression of the, bioluminescent gene wherein the gene set includes the regulatory gene nagR and a promoter region inducing the transcription of the bioluminescent gene via the action of protein NagR encoded by the gene nagR. Further disclosed are a transformant containing the recombinant vector and a method for detecting benzoic acid and derivatives, thereof by measuring bioluminescence generated after reacting the transformant with a sample to be tested.

### RELATED APPLICATIONS

This application claims the benefit of priority of Korean Patent Application No. 2003-0034915, filed on May 30, 2003, the entire disclosure of which is expressly incorporated herein by reference in its entirety.

### BACKGROUND OF THE INVENTION

1. Field of the Invention

The present invention relates to a recombinant vector for transforming a strain to detect benzoic acid and derivatives thereof, transformant containing the recombinant vector, and a method for detecting benzoic acid and derivatives thereof using the transformant. More particularly, the present invention relates to a recombinant vector for transforming a strain to detect benzoic acid and derivatives thereof which can determine to what degree soil contaminated with polyaromatic hydrocarbons has been recovered, a transformant containing the recombinant vector, and a method for detecting benzoic acid and derivatives thereof by measuring bioluminescence generated after reacting the transformant with a sample to be tested.

2. Description of the Related Art

Since aromatic compounds are stable in view of their structure, they are limitedly biodegradable in nature. For this reason, the aromatic compounds in soil cause serious soil contamination. Polyaromatic hydrocarbons, non-biodegradable compounds, are partially biodegraded by microorganisms present in soil and can be used as carbon sources. Thus, studies on biological methods for recovering contaminated soil using soil microorganisms are being actively undertaken.

Since benzoic acid and derivatives thereof are aromatic compounds obtained as intermediate metabolites during natural degradation of polyaromatic compounds, the analysis of benzoic acid and derivatives thereof in the course of recovery of soil contaminated with polyaromatic hydrocarbons facilitates the determination of the degree of soil recovery.

Conventional instrumental analyses are advantageous in that they can provide quantitative analysis of chemical data, but have disadvantages that the toxicity of chemicals to be analyzed cannot be environmental toxicologically measured and analysis on intracellular reaction cannot be performed.

Trials to monitor and classify the toxicity of chemicals using bioluminescent bacteria are currently ongoing. For example, a method for determining the degree of a certain stress caused by a specific chemical as bioluminescence intensity was reported (M. B. Gu and S. H. Choi, Water Science and Technology, 43: 147-154). According to this method, a bioluminescent gene is linked to a stress promoter which can induce the expression of a bioluminescent substance upon causing stress. In addition, a protein-coding gene involved in the intermediate metabolism associated with the degradation of a specific chemical, and a promoter inducing expression of the gene are used for the detection of the chemical (Burlage, R. S., Sayler, G. S., Larimer, F, J. Bacteriol. 172:4749-4757). Accordingly, the production of bioluminescent bacteria using gene recombination techniques can be broadly applied to the biological testing methods of chemicals.

However, despite many advantages of biological testing methods described above, no technology has been established that provides methods for analyzing benzoic acid and derivatives thereof generated during recovery of soil contaminated with polyaromatic hydrocarbons.

### SUMMARY OF THE INVENTION

Therefore, the present invention has been made in view of the above problems, and it is an object of the present invention to provide a recombinant vector for transforming a strain to detect benzoic acid and derivatives thereof which can determine to what degree soil contaminated with polyaromatic hydrocarbons has been recovered.

It is another object of the present invention to provide a transformant produced by transforming Escherichia coli with the recombinant vector.

It is yet another object of the present invention to provide a method for detecting benzoic acid and derivatives thereof by measuring bioluminescence generated after reacting the transformant with a sample to be tested.

In accordance with one aspect of the present invention, there is provided a recombinant vector for transforming a strain to detect benzoic acid and derivatives, comprising:

a bioluminescent gene encoding a bioluminescent protein; and

a gene set inducing the expression of the bioluminescent gene,

wherein the gene set includes the regulatory gene nagR and a promoter region inducing the transcription of the bioluminescent gene via the action of protein NagR encoded by the gene nagR.

In accordance with another aspect of the present invention, there is provided a transformant produced by transforming E. coli with the recombinant vector.

In accordance with yet another aspect of the present invention, there is provided a method for detecting benzoic acid and derivatives thereof by measuring bioluminescence generated after reacting the transformant with a sample to be tested.

The regulatory protein NagR expressed by the nagR gene binds to a promoter which operates nag operon, i.e., genes comprising nagAaGHAbAcAdBFCQED, involved in the conversion of naphthalene to gentisate to regulate the transcription of the nag operon (S. L. Fuenmayor, M. Wild, A. L. Boyes, and P. A. Williams, J. Bacteriol. 180: 2522-2530).

The promoter region of the nag operon is designated as ‘PnagG’, whose operation is regulated by the regulatory protein NagR expressed by the nagR gene. The main mechanism is explained based on the observation that when the regulatory protein bound with benzoic acid and derivatives thereof binds to the promoter region, it affects the expression of the bioluminescent gene.

The gene set which induces the expression of the bioluminescent gene includes the nagR gene and the PnagG, a promoter region of the nag operon. The gene set (nagR-P_{nagG}) functions like a single promoter, and can be obtained by PCR amplification of 5′ primer (5′-GTCACCAATATGGACCAGGCAACGC-3′) shown in SEQ ID NO: 1 and 3′ primer (5′-CCGCGTCTAGATGCTAATTGAGGGG-3′) shown in SEQ ID NO: 2 derived from Ralstonia sp. U2. The PCR product thus obtained is treated with a particular restriction enzyme and is then inserted into a specific vector previously treated with the same restriction enzyme to produce a recombinant vector. Thereafter, the recombinant vector is recombined with a specific vector carrying a bioluminescent gene to produce a recombinant vector for transforming the E. coli to detect benzoic acid and derivatives thereof according to the present invention.

### BRIEF DESCRIPTION OF THE DRAWINGS

The above and other objects, features and other advantages of the present invention will be more clearly understood from the following detailed description taken in conjunction with the accompanying drawings.

FIG. 1 is a diagram showing a method for producing a recombinant vector (PNAG1) according to the present invention.

FIG. 2 is a photograph showing the result determined by agarose gel electrophoresis after cleaving a recombinant vector (pNAG1) of the present invention pNAG1 with restriction enzymes EcoR1 and Kpn1 (left lane: marker, right lane: vector fragment).

FIGS. 3a and 3b are graphs showing changes in the bioluminescence after treating a transformant (EBNAG1) of the present invention with benzoic acid (a) and salicylic acid (b), as a benzoic acid derivative, at various concentrations, respectively.

### DETAILED DESCRIPTION OF THE PREFERRED EMBODIMENT

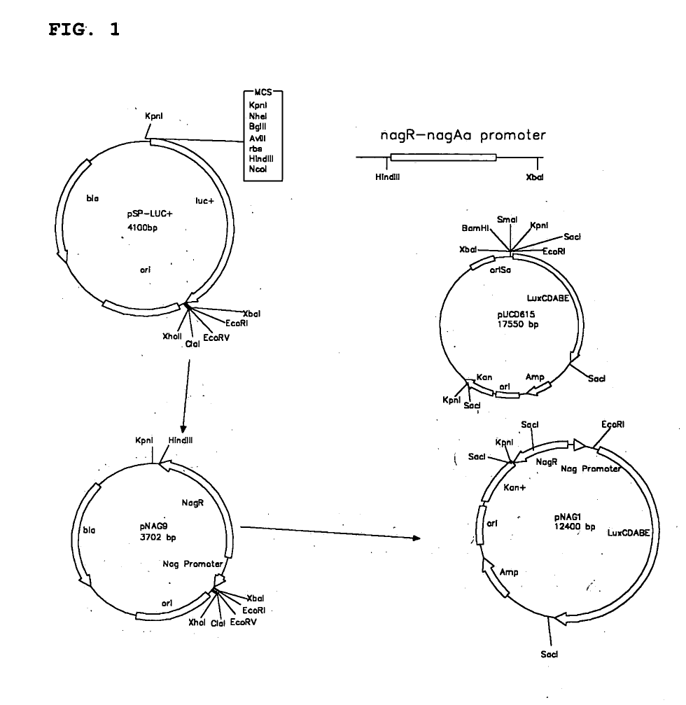

Hereinafter, the present invention will be explained in more detail with reference to the accompanying drawings. FIG. 1 is a cleavage map showing a method for producing a recombinant vector for transforming E. coli to detect benzoic acid and derivatives thereof, in accordance with a preferred embodiment of the present invention. Referring to FIG. 1, recombinant plasmid pNAG1 is produced by fusing a promoter containing the nagR gene and P_{nagG} to lux CDABE, a bioluminescent gene. The nagR gene derived from Ralstonia sp. U2 promotes the catabolism of naphthalehe, and the P_{nagG} is a promoter of the nag operon.

Primers shown in SEQ ID NOs: 1 and 2 from the genome of Ralstonia sp. U2 are amplified to obtain a 1.331-kb PCR product shown in SEQ ID NO: 3. The PCR product includes a −266 bp downstream section and a +176 bp upstream section of the nagR gene, and contains promoter regions of nagR [267˜1172 bp] and nagAa gene [1173˜1288 bp]. The PCR product is treated with restriction enzymes Xba1 and HindIII, and then recombined to pSP-luc+ previously treated with the same enzymes to produce a recombinant plasmid designated as ‘pNAG9’.

The pNAG9 plasmid is treated with restriction enzymes Kpn1 and EcoR1, and is then recombined to the multicloning site of pUCD615 which contains luxCDABE treated with the same restriction enzymes, to produce a recombinant plasmid designated as ‘pNAG1’. Preferably, the recombinant plasmid pNAG1 contains an antibiotic resistance gene. Examples of the antibiotic resistance gene used in the present invention include many known genes, e.g., kanamycin-, ampicillin-, tetracycline-resistance genes, etc. Accordingly, a person skilled in the art can appropriately select the desired gene from these antibiotic resistance genes, but the constitution of the present invention is not limited to the specific kind of the genes. Since the antibiotic resistance gene is introduced for the selection of a desired transformant, any genes that are used for the selection can be introduced. Accordingly, genes introduced into the recombinant plasmid pNAG1 are not limited to these antibiotic resistance genes.

In the following examples of the present invention, the E. coli RFM443 strain is used as a microorganism transformed by the recombinant plasmid pNAG1. E. coli is most suitably used in terms of simple culturing conditions and easy manipulation, but the present invention is not limited thereto. The pNAG1 plasmid carrying the kanarycin- or ampicillin-resistance gene is introduced into the E. coli RFM443, and then grown on an LB medium supplemented with ampicillin, from which colonies containing the plasmid pNAG1 can be screened.

The present inventors screened a strain showing bioluminescence response to benzoic acid and salicylic acid as a representative benzoic acid derivative, and designated it as ‘E. coli RFM443/pNAG1 (EBNAG1)’. The E. coli RFM443/pNAG1 (EBNAG1) was deposited on Apr. 4, 2003 with the Korean Agricultural Culture Collection (KACC) under the accession No. KACC 91044.

When the transformant EBNAG1 (KACC 91044) of the present invention detects the presence of benzoic acid and derivatives thereof, the bioluminescence intensity increases. Accordingly, the measurement of bioluminescence generated after exposing the strain of the present invention to a sample to be tested enables not only the detection of benzoic acid and derivatives thereof as representative soil contaminants, but also the detection of the toxicity and harmfulness of benzoic acid and derivatives thereof present in water.

Hereinafter, the present invention will be described in more detail with reference to the following examples. However, these examples are given for the purpose of illustration and are not to be construed as limiting the scope of the invention.

### EXAMPLE 1

### Production of Recombinant Plasmid

A 1.33 kb PCR product, i.e. nagR-P_{nagG} promoter, was obtained by PCR amplification of primers shown in SEQ ID NOs: 1 and 2 derived from Ralstonia sp. U2. Since the PCR product requires restriction enzyme sites of Kpn1 and EcoR1 to combine to pUCD615 as a terminal vector, pSP-luc+ (Promega, USA) having restriction enzyme sites of Kpn1 and EcoR1 was used. At this time, the PCR product and the vector pSP-luc+ were treated with restriction enzymes Xab1 and HindIII at 37°X for 2 hours, respectively, to remove the luc gene from the vector, and were then recombined to each other to produce a recombinant plasmid (pNAG9).

The plasmid pNAG9 was treated with restriction enzymes Kpn1 and EcoR1 at 37° C. for 2 hours, and recombined to multicloning sites of pUCD615 containing luxCDABE previously treated with the same restriction enzymes to produce a recombinant plasmid designated as ‘pNAG1’ (FIG. 1).

### EXAMPLE2

### Production of Transformant EBNAG1

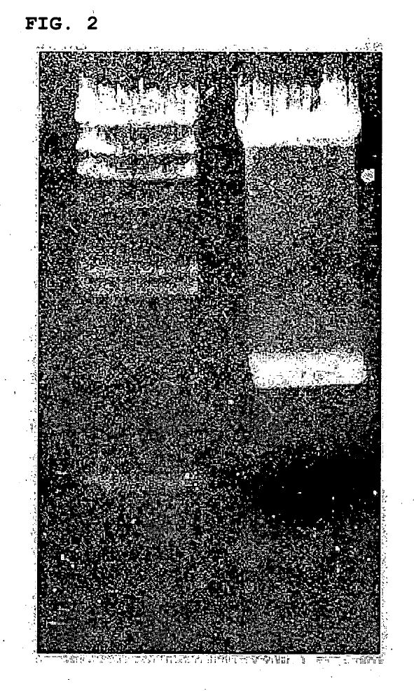

First, wild-type strain RFM443 was cultivated at 37° C. for 1 day. Salt ingredients present in the bacterial culture were removed using 50% glycerol, and the strain was then electroporated to produce viable host cells. The plasmids produced in Example 1 were inserted into the host cells, and subjected to electroporation in an electroporation system (Bio-RAD, Gene Pulser^{R} II) for 2 seconds. The electroporated host cells were spread onto an LB-agar plate supplemented with 50 μg/ml ampicillin. The plate was placed in an incubator at 30° C. and incubated for 1 day to form colonies. The colonies were seeded onto a 100 ml LB medium supplemented with 50 μg/ml ampicillin, transferred into a rotary incubator at 30° C., and incubated for 1 day. Thereafter, recombinant plasmid (PNAG1) was isolated from the culture using a commercially available Miniprep kit (Qiagen). The plasmids were treated with restriction enzymes Kpn1 and EcoR1, which was previously used to produce the recombinant plasmid in Example 1, to identify the production of the plasmid, and 11-kb pUCD615 vector and a 1.06 kb promoter region were then identified using a 0.8% agarose gel (FIG. 2).

### EXAMPLE3

### Determination of Detectability of Transformant EBNAG1 on Benzoic Acid and Derivatives Thereof

The EBNAG1 strain was cultivated in a 100 ml LB medium supplemented with 50 μg/ml ampicillin at 30° C. until the absorbance reached 0.08 (at 600 nm). 100 μl of aliquot was taken from the culture for the measurement of the bioluminescence intensity, and was then poured on each well of an opaque96-well plate [Microlite™, Thermo Labsystems, USA] containing chemicals to be tested (benzoic acid concentrations: 12.5, 6.25, 3.125, 1.5625, 0.78125, 0.390625 and 0.1953125 mM, and salicylic acid concentrations: 12.5, 6.25, 3.125, 1.5625, 0.78125, 0.390625 and 0.1953125 mM). The well plates were placed in a 96-well microtiter plate reader (MLX Microtiter® Plate Luminometer, DYNEX Technology, USA) to measure the bioluminescence intensity.

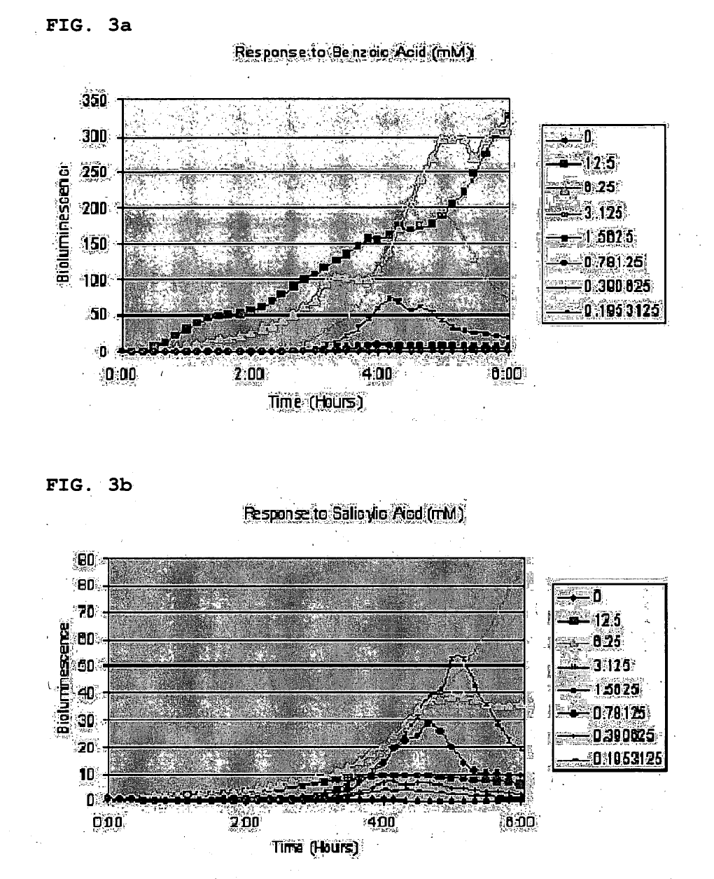

Then, changes in the bioluminescence intensity were observed for 6 hours at varying chemical concentrations. FIGS. 3a and 3b show the results of bioluminescent signals obtained from EBNAG1 after seeding benzoic acid and salicylic acid as a representative benzoic acid derivative at various concentrations, respectively. As can be seen from the graph of FIG. 3a, the bioluminescence intensity varied with increasing benzoic acid concentrations 3 hours after seeding. It was also observed that the bioluminescence intensity increased in proportion to the benzoic acid concentrations. In addition, as can be seen from the graph of FIG. 3b, the bioluminescence intensity varied with increasing salicylic acid concentrations 3 hours after seeding. It was also observed that the bioluminescence intensity increased in proportion to the salicylic acid concentrations. These results suggest that the recombinant plasmid pNAG1 can be used to detect the toxicity of a chemical present in a sample, as well as the toxicity depending on the concentrations of benzoic acid and derivatives thereof. The degrees of EBNAG1 response to benzoic acid and other benzoic acid derivatives are shown in Table 1 below.

TABLE 1

| | Maximum RBL^{a} | |
|---|---|---|
| Compounds | (Concentration) | MDC^{b}(mM) |
| Benzoic acid | 344 (12.5 mM) | 0.39 |
| Salicylic acid | 51.2 (6.25 mM) | 0.195 |
| 4-Chlorosalicylic acid | 92.7 (1.56 mM) | <0.39 |
| 5-Chlorosalicylic acid | 8.8 (1.56 mM) | 0.39 |
| 2,4-Dihydroxybenzoic acid | 11.4 (6.25 mM) | 3.13 |
| 3,4-Dihydroxybenzoic acid | 10.9 (6.25 mM) | 6.25 |
| 3,5-Dihydroxybenzoic acid | 13.0 (12.5 mM) | 3.13 |
| 3,4-Dimethoxybenzylalcohol | 4.92 (6.25 mM) | 0.39 |

RBL^{a}(Relative bioluminescence) represents a value calculated by dividing the bioluminescence of a sample by the bioluminescence of a control.
MDC^{b}(Minimum detection concentration) represents the minimum concentration corresponding to an RBL of 2.

According to the present invention, the toxicity and harmfulness of benzoic acid as an aromatic compound and derivatives thereof present in soil can be analyzed without the use of complex instruments. Since benzoic acid and derivatives thereof are aromatic compounds obtained as intermediate metabolites during natural degradation of polyaromatic compounds, the analysis of benzoic acid and derivatives thereof in the course of recovery of soil contaminated with polyaromatic hydrocarbons facilitates the determination of the degree of soil recovery.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A recombinant vector for transforming a strain to detect benzoic acid and derivatives, comprising:
a bioluminescent gene encoding a bioluminescent protein; and
a gene set inducing the expression of the bioluminescent gene, wherein the gene set includes regulatory gene nagR and a promoter region inducing the transcription of the bioluminescent gene via the action of protein NagR encoded by the gene nagR.

2. The recombinant vector according to claim 1, wherein the promoter region inducing the transcription of the bioluminescent gene is a promoter region of the nag operon.

3. The recombinant vector according to claim 1 or 2, wherein the bioluminescent gene is luxCDABE gene.

4. The recombinant vector according to claim 1, wherein the recombinant plasmid has a cleavage map shown in FIG. 1, and is pNAG1 for transforming E. coli to detect benzoic acid and derivatives.

5. The recombinant vector according to claim 4, wherein the E. coli is E. coli RFM 443.

6. Transformant EBNAG1 (KACC 91044) produced by transforming E. coli with the recombinant vector according to claim 4.

7. A method for detecting benzoic acid and derivatives thereof by measuring bioluminescence generated after reacting the transformant according to claim 6 with a sample to be tested.
